(19) United States
(12) Patent Application Publication
(10) Pub. No.: US 2014/0330164 A1
(43) Pub. Date: Nov. 6, 2014

(54) ENDOSCOPIC TREATMENT TOOL

(71) Applicant: OLYMPUS MEDICAL SYSTEMS CORP., Tokyo (JP)

(72) Inventor: Keita SUZUKI, Tokyo (JP)

(21) Appl. No.: 14/264,313

(22) Filed: Apr. 29, 2014

Related U.S. Application Data
(60) Provisional application No. 61/818,015, filed on May 1, 2013.

(51) Int. Cl. A61B 10/06; A61B 10/04

(52) U.S. Cl. CPC A61B 10/06; A61B 10/04; A61B 2010/045
USPC 600/567

(57) ABSTRACT

An endoscopic treatment tool includes: an elongated flexible insertion part, a treatment part which is disposed at a distal end part of the insertion part, a first operation member which is attached to the treatment part and is moved to the insertion part so as to operate the treatment part, a second operation member of which a distal end is attached to the first sheath, a third sheath formed with a smaller diameter than the first sheath, an operation part main body which is attached to a proximal end part of the third sheath, a first operation part which is provided in the main body of the operation part and attached to a proximal end part of the first operation member, and a second operation part which is provided in the operation part main body and attached to a proximal end part of the second operation member.

### BACKGROUND OF THE INVENTION

1. Field of the Invention

The present invention relates to an endoscopic treatment tool, and more specifically relates to an endoscopic treatment tool including an insertion part, a part of which is with an adjustable rigidity.

Priority is claimed on US Provisional Patent Application No. 61/818,015, filed on May 1, 2013, the content of which is incorporated herein by reference.

2. Description of Related Art

A conventionally known flexible endoscopic treatment tool is configured that a treatment part which is used in treating a body tissue is provided at a distal end of a flexible elongated insertion part of the flexible endoscopic treatment tool.

For example, Japanese Unexamined Patent Application, First Publication H09-299378 discloses an endoscopic treatment tool which is configured with a pair of forceps members provided on a treatment part of the endoscopic treatment tool. The pair of forceps members are connected to an operation member such as an operation wire via a link mechanism so that the pair of forceps members are capable of being opened by advancing the operation member toward the treatment part and being closed by retracting the operation member. A coil sheath is used as an insertion part. According to the endoscopic treatment tool, since the treatment part is attached at the distal end of the insertion part, when the operation member is retracted, the coil sheath of the insertion part is compressed in an axial direcetion so that rigidity of the endoscopic treatment tool increases as well as flexibility of the endoscopic treatment tool decreases.

### SUMMARY OF THE INVENTION

According to a first aspect of the present invention, an endoscopic treatment tool includes an elongated flexible insertion part which has a first sheath formed by a coil and provided at a distal end side of the insertion part, a second sheath provided at a proximal end side of the insertion part, and a connecting member provided so as to communicate the first sheath with the second sheath, a treatment part which is disposed at a distal end part of the insertion part and used for an intracorporal treatment, a first operation member which is attached to the treatment part and is moved to the insertion part so as to operate the treatment part, a second operation member of which a distal end is attached to the first sheath, a third sheath formed with a smaller diameter than the first sheath, a distal end of the third sheath being connected to the connecting member, the second operation member being inserted through the third sheath, an operation part main body which is attached at a proximal end part of the third sheath, a first operation part which is provided in the operation part main body and attached to a proximal end part of the first operation member so as to move the first operation member to the insertion part, and a second operation part which is provided in the operation part main body and attached to a proximal end part of the second operation member so as to move the second operation member to the insertion part.

### BRIEF DESCRIPTION OF THE DRAWINGS

FIG. 1 is an overall diagram illustrating an entire configuration of a grasping forceps as an endoscopic treatment tool according to a first embodiment of the present invention.

FIG. 2 is an enlarged partial cross section of a vicinity of a treatment part of the grasping forceps according to the first embodiment of the present invention.

FIG. 3 is an enlarged partial cross section of the vicinity of a connecting member of the grasping forceps according to the first embodiment of the invention.

FIG. 4 is an enlarged partial cross section of a distal end part of an operation part of the grasping forceps according to the first embodiment of the present invention.

FIG. 5 is an enlarged partial cross section of a vicinity of a treatment part of the endoscopic treatment tool according to a second embodiment of the present invention.

FIG. 6 is a diagram illustrating an operation performed by the treatment part when using the endoscopic treatment tool according to the second embodiment of the present invention.

FIG. 7 is a diagram of a distal end part of the endoscopic treatment tool according to the second embodiment of the present invention.

### DETAILED DESCRIPTION OF THE INVENTION

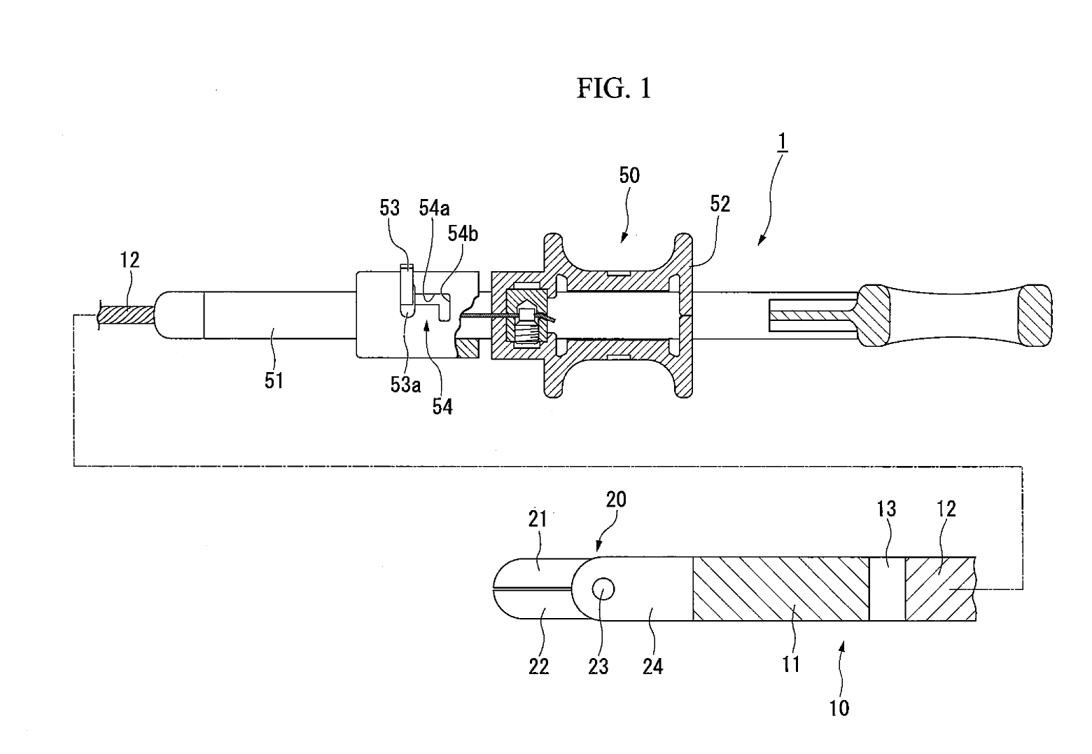

Hereinafter, a first embodiment of the present invention will be described with reference to FIG. 1 to FIG. 4.

FIG. 1 is an overall diagram illustrating an entire configuration of grasping forceps 1 used as an endoscopic treatment tool according to a first embodiment of the present invention. The grasping forceps 1 includes an elongated flexible insertion part 10, a treatment part 20 which is attached at a distal end of the insertion part 10, and an operation part 50 which is attached at a proximal end of the insertion part 10.

The insertion part 10 is configured such that a first coil-sheath (coil-sheath) 11 being on a side of the treatment part 20 and a second coil-sheath (second sheath) 12 being on a side of the operation part 50 are connected to a connecting member 13 so that the coil-sheath 11 and the second sheath 12 communicate with each other. A length of the first coil-sheath 11 is capable of being set appropriately. However, as described below, since rigidity of the first coil sheath 11 is capable of being increased temporarily by performing a predetermined operation, it is preferred that a length of the first coil-sheath 11 is set to a value so that only the first coil-sheath 11 is capable of being outside from the endoscope, when the grasping forceps 1 protrude from a distal end of the endoscope by a maximum length during an intracorporal treatment.

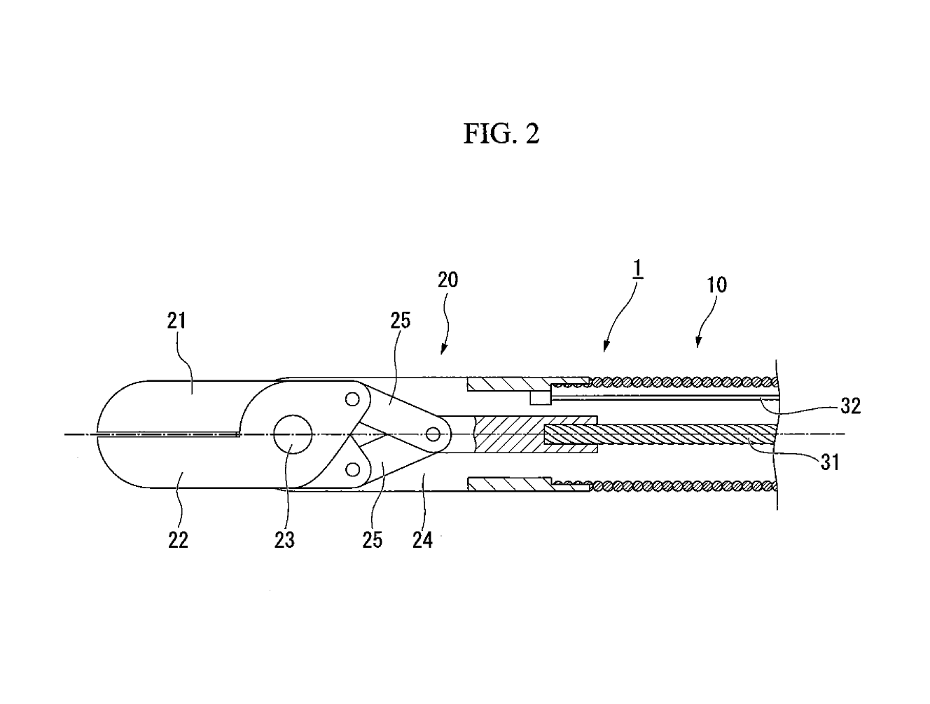

FIG. 2 is an enlarged partial cross section of a vicinity of a treatment part 20. The treatment part 20 is configured with a pair of forceps pieces 21 and 22 which have the same shape and the same size, and the pair of forceps pieces 21 and 22 are connected at a rotating axis 23 so as to be rotatable relative to each other. The rotating axis 23 is fixed to a cover 24 which is fixed to the distal end of the insertion part 10 so that the rotating axis 23 is fixed to the insertion part 10. Each of proximal ends of the forceps pieces 21, 22 is connected to a first operation wire (first operation member) 31 via a link member 25. The first operation wire 31 is inserted through the insertion part 10 and connected to the operation part 50. A distal end of a second operation wire (a second operation member) 32 is fixed to an inside surface of the cover 24, using welding or the like.

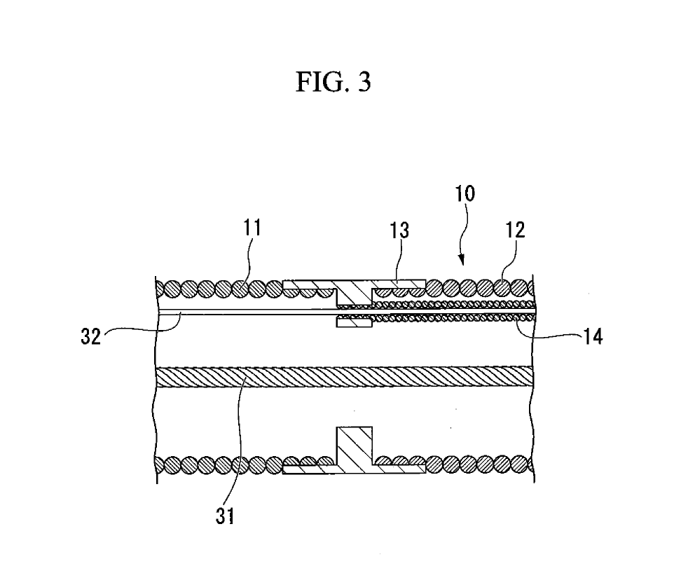

FIG. 3 is an enlarged partial cross section of a vicinity of a connecting member 13. The proximal end of the first coil sheath 11 and the distal end of the second coil sheath 12 are fixed to a distal end and a proximal end of the connecting member 13, respectively, using welding, adhering, or the like. The second coil sheath 12 is inserted through by a third coil sheath (third sheath) 14 with a smaller diameter than the first coil sheath 11 and the second coil sheath 12. A distal end of the third coil sheath is fixed to the connecting member 13 at a position inside the insertion part 10. The second operation wire 32 is inserted through the third coil sheath 14. The third coil sheath 14 and the second operation wire 32 extend to the operation part 50.

As illustrated in FIG. 1, the operation part 50 includes an operation part main body 51 which is attached at the proximal end of the insertion part, a slider (first operation part) 52 which is provided so as to be capable of sliding in a longitudinal direction of the operation part main body 51, and a sheath operation lever (second operation part) 53 which is attached to the operation part main body 51.

A proximal end of the first operation wire 31, which extends through the insertion part 10, protrudes to the inside of the operation part main body 51 and is fixed to the slider 52. Accordingly, the first operation wire 31 is capable of protruding/retracting to the insertion part 10 by sliding the slider 52 to the operation part main body 51.

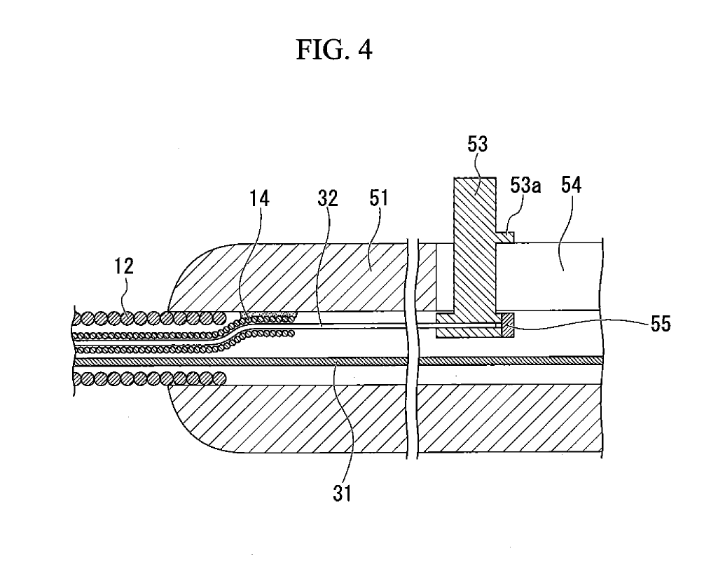

FIG. 4 is an enlarged partial cross section of a distal end part of an operation part. The proximal end of the second coil sheath protrudes to the inside of the operation part main body 51, but is not fixed to the operation part main body 51. The proximal end of the third coil sheath 14 protrudes from the proximal end of the second sheath 12 and is fixed to the interior surface of the operation part main body 51. The second operation wire 32 which protrudes from the proximal end of the third coil sheath 14 is fixed to the sheath operation lever 53.

The sheath operation lever 53 is inserted through a groove 54 which extends through the operation part main body 51 so as to be capable of being moved along the groove 54. The groove 54 includes a traction groove 54a and a lock groove 54b. The traction groove 54a extends in a longitudinal direction of the operation part main body 51. The lock groove 54b communicates with the traction groove 54a and extends in a circumferential direction of the operation part main body 51. The sheath operation lever 53 is retracted along the traction groove 54a so that the second operation wire 32 is capable of being drawn to a side of the proximal end of the operation part 50. The sheath operation lever 53 is moved to be coupled into the lock groove 54b so that the second operation wire 32 is capable of retaining a state of being drawn. The sheath operation lever 53 is supported by a flange 53a and a stopper 55 so as to not to be drawn out from the groove 54.

An operation performed when using the grasping forceps 1 with the above configuration according to the present embodiment will be explained.

Firstly, a user inserts an endoscope (not shown) into a patient's body, and causes the distal end of the endoscope to be moved to a vicinity of an intracorporal treatment target tissue. Then, the user inserts the grasping forceps 1 into a forceps channel from a side of the treatment part side to cause the treatment part 20 to protrude from a distal opening of the forceps channel.

The user advances the slider 52 of the operation part 50 so as to open the pair of the forceps 21, 22, while observing the treatment target tissue in a field of view of the endoscope. When a part of the treatment target tissue is positioned between the pair of the forceps pieces 21, 22, the slider 52 is retracted to close the pair of the forceps pieces 21, 22 so as to grasp the target tissue.

When the insertion part 10 of the grasping forceps 1 is desired to protrude long (for example, protrude with a length equal to 10 centimetres or longer), since the insertion part 10 is flexible, the treatment part 20 hangs down in a direction that the gravity applies. Accordingly, in some cases, it is difficult to cause the insertion part 10 to protrude long in a direction parallel to an axial direction of the insertion part. In such cases, the user retracts the sheath operation lever 53 along the traction groove 54a so that the second operation wire 32 is drawn back to the insertion part 10. As a result, the first coil sheath 11 and the third coil sheath 14 are compressed in the axial direction of the insertion part 10 by a force applied to the cover 24 to cause the cover 24 to move toward the proximal end side of the insertion part 10. Since the proximal end of the second coil sheath 12 is not fixed to the operation part main body 51, the second coil sheath 12 is not compressed even when the second operation wire 32 is drawn.

Since the first coil sheath 11 is compressed in the axial direction, compared to an un-compressed state, rigidity of the first coil sheath 11 increases and flexibility of the first coil sheath 11 decreases. As a result, it is easier for the first coil sheath 11 to retain a linear state and more difficult to be bent, even when the first coil sheath 11 is protruded from the endoscope.

Regarding a region of the insertion part 10 closer to the proximal end than the connecting member 13, the third coil sheath 14 is compressed in the axial direction. However, since the third coil sheath 14 has a smaller diameter than the first coil sheath 11 and since the second coil sheath 12 is not compressed, the degree of rigidity increase of the third coil sheath 14 is far lower than a region of the insertion part 10 closer to the distal end of the insertion part 10 than the connecting member 13. Accordingly, even the second operation wire 32 is drawn, the flexibility of the region closer to the proximal end of the insertion part 10 than the connecting member 13 is difficult to decrease.

When the sheath operation lever 53 is moved into the lock groove 54b, even when the user removes his/her hand from the sheath operation lever 53, it is possible to retain the second operation wire 32 to be drawn. When it is desired to restore the rigidity of the insertion part 10 to the initial value, the user can move the sheath operation lever 53 outside from the lock groove 54b and advance the sheath operation lever 53 along the traction groove 54a so as to release the traction by the second operation wire 32.

According to the endoscopic treatment tool 1 of the present embodiment, an operation of the operation part 20 and an alteration of the rigidity of the insertion part 10 are capable of being employed independently. That is, the first operation wire 31 used for operating the operation part 20 is connected to the slider 52 of the operation part 50, and the second operation wire 32 used for adjusting the rigidity of the insertion part 10 is connected to the sheath operation lever 53 of the operation part 50. Since the movement of the treatment part 20 and the alteration of the rigidity of the insertion part 10 are capable of being employed independently, it is possible to open/close the pair of forceps pieces 21, 22 to carry out treatment while the insertion part 10 is in a state that the rigidity of the insertion part 10 is increased.

In the region closer to the proximal end of the insertion part 10 than the connecting member 13, the third coil sheath 14 with a smaller diameter than the first coil sheath 11 is compressed. Accordingly, a force used for compressing the region closer to the proximal end of the insertion part 10 than the connecting member 13 is decreased, and rigidity increase of the region closer to the proximal end of the insertion part 10 than the connecting member 13 is suppressed. As a result, it is possible to selectively increase the rigidity of the region closer to the distal end of the insertion part 10 than the connecting member 13 by using a smaller operating force.

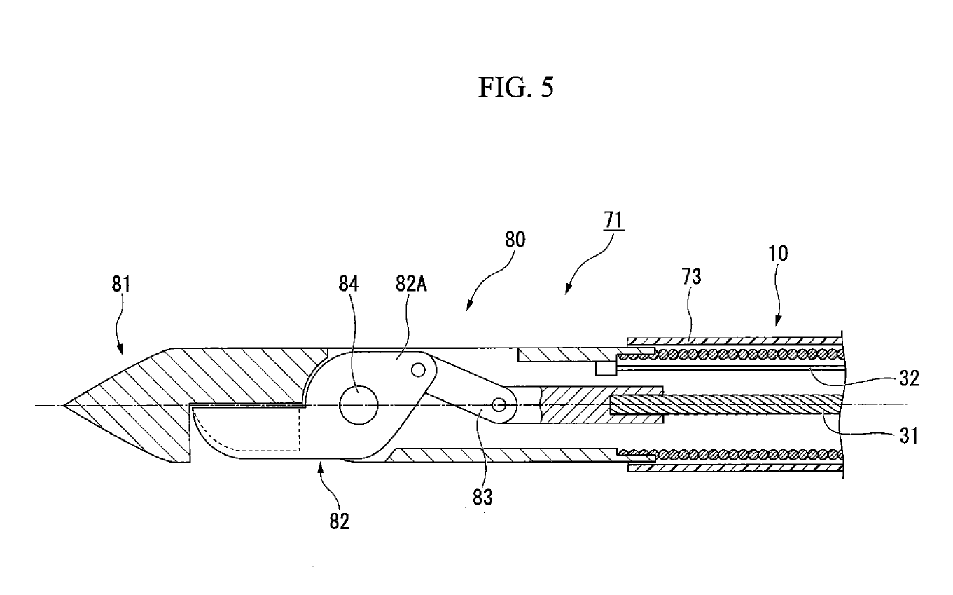

Subsequently, a second embodiment of the present invention will be explained with reference to FIGS. 5 to 7. A difference between a treatment tool 71 of this embodiment and the treatment tool 1 of the first embodiment is a configuration of the treatment part and the second operation part. In the following explanation, constitutive elements which are common to the treatment tool 1 according to the first embodiment are designated with like reference numerals and are not repetitiously explained.

FIG. 5 is an enlarged partial cross section of a vicinity of a treatment part 80 of the endoscopic treatment tool 71 according to a second embodiment of the present invention. The treatment part 80 includes a puncture part 81 and a collecting member 82. A distal end of the puncture part 81 is formed sharply so as to be punctured into the tissue. The collecting member 82 is configured to be capable of rotating to the puncture part 81. That is, the endoscopic treatment tool 71 according to this embodiment is used in a combination with an ultrasonic endoscope or the like as a biopsy needle. A distal end of the collecting member 82 is formed in a cup shape so as to be capable of containing the tissue. A proximal end 82A of the collecting member 82 is connected to the first operation wire 31 via a link member 83. The treatment part 80 is inserted into the forceps channel of the endoscope in a state of being accommodated in an outer sheath 73 so as to prevent the puncture part 81 from hurting an interior surface of the forceps channel of the endoscope and is protruded from the outer sheath 73 during a biopsy.

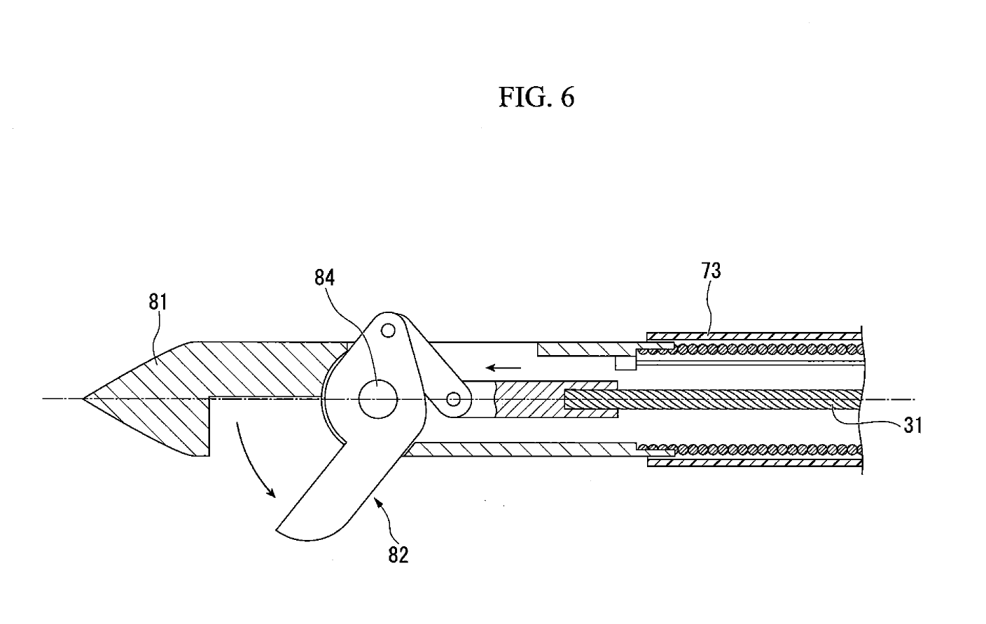

When the treatment part 80 is used, firstly, the puncture part 81 is punctured into an organ from which a tissue is collected. Then, as illustrated in FIG. 6, the slider 52 is advanced so that the collecting member 82 rotates on a rotating axis 84 and protrudes from the outer peripheral surface of the puncture part 81. In this state, the slider 52 is retracted so that part of the tissue is scraped from the organ by the collecting member 82 and accommodated in the cup-shaped space provided in the distal end of the collecting member 82, while the collecting member 82 is accommodated in the puncture part 81.

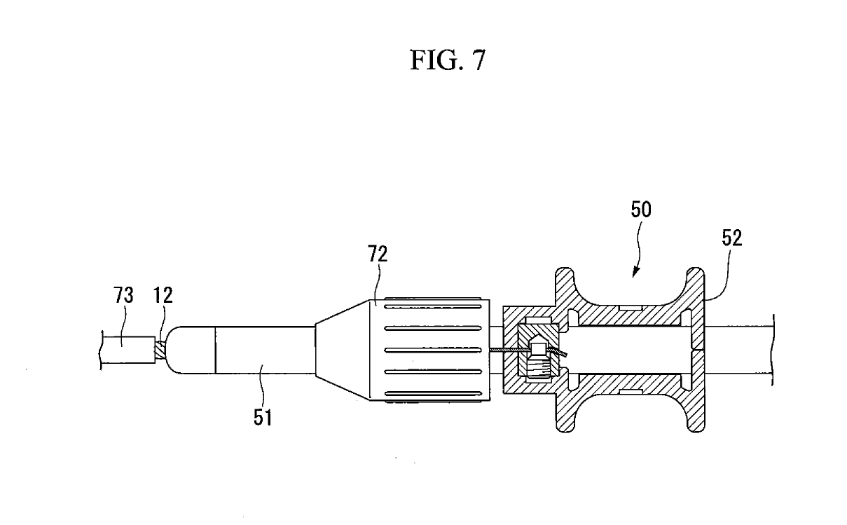

FIG. 7 is an enlarged partial diagram of an operation part 50 of the endoscopic treatment tool 71. According to this embodiment, a sheath operation dial 72 is provided in the operation part main body 51 instead of the sheath operation lever 53. Regarding a point that the sheath operation dial 72 is rotated so that the second operation wire 32 is capable of advancing/retracting, the present embodiment is the same as the first embodiment. Further, since the sheath operation dial 72 engages with the operation part main body 51 when the sheath operation dial 72 is rotated by a predetermined degree (for example, 45 degrees), an advancing/retracting state of the second operation wire 32 is capable of being retained even when the user removes his/her hand from the sheath operation dial 72.

In the endoscopic treatment tool 71 according to this embodiment, it is possible to selectively increase the rigidity of the first coil sheath 11 by rotating the sheath operation dial 72 to draw the second operation wire 32.

When the puncture part provided in the distal end of the biopsy needle such as the endoscopic treatment tool 71 is used to puncture the organ, the region of the insertion part that protrudes from the endoscope is required with high rigidity. Particularly, in a situation in which a fibrosis arises in the treatment organ that the organ becomes rigid, since the rigidity of the region of the insertion part that protrudes from the endoscope increases greatly, it is possible to puncture the organ favorably and collect the tissue favorably.

According to this embodiment, a length of the first coil sheath 11 whose rigidity increases greatly is favorably, for example, in a situation in which the treatment target organ is an organ of the liver and gall pancreas system such as the liver, the gallbladder, or the pancreas, the length of the first coil sheath 11 is substantially 6-8 centimeters, in a situation in which the insertion part 10 is inserted into the bronchi for collecting a sample of the bronchi tissue or the lymph node tissue, the length of the first coil sheath 11 is substantially 3-4 centimeters.

For example, in the treatment tool according to the present invention, a plurality of the second operation members may be provided. The plurality of the second operation members are disposed at even intervals in a circumferential direction of the insertion part so that the compression force by the second operation members apply to the insertion part uniformly. Accordingly, it is possible to favorably make the adjustment of the rigidity of the insertion part.

Further, since the rigidity of the second sheath and the third sheath may not always necessary to be increased by drawing the second operation member, the second sheath and the third sheath may be formed of a material such as a resin tube or the like, other than a coil.

Further, according to the present invention, the treatment part of the endoscopic treatment tool is not limited to the above-mentioned examples. For example, the treatment part may be a treatment part which is supplied with high-frequency current. Also, regarding the treatment part provided with a pair of forceps pieces, each of the forceps pieces may be connected with the first operation member so that two of first operation members are provided. In addition, a treatment tool with any known structure may be appropriately selected and employed.

While preferred embodiments of the present invention have been described above, these are not limitative of the invention. Additions, omissions, substitutions, and other modifications can be made without departing from the spirit or scope of the present invention. Accordingly, the present invention is not to be considered as being limited by the foregoing description, and is only limited by the scope of the appended claims.

What is claimed is:

1. An endoscopic treatment tool comprising:
an elongated flexible insertion part which has a first sheath formed by a coil and provided at a distal end side of the insertion part, a second sheath provided at a proximal end side of the insertion part, and a connecting member provided so as to communicate the first sheath with the second sheath;
a treatment part which is disposed at a distal end part of the insertion part and used for an intracorporal treatment;
a first operation member which is attached to the treatment part and is moved to the insertion part so as to operate the treatment part;
a second operation member of which a distal end is attached to the first sheath;
a third sheath formed with a smaller diameter than the first sheath, a distal end of the third sheath being connected to the connecting member, the second operation member being inserted through the third sheath;
an operation part main body which is attached to a proximal end part of the third sheath;
a first operation part which is provided in the operation part main body and attached to a proximal end part of the first operation member so as to move the first operation member to the insertion part; and
a second operation part which is provided in the operation part main body and attached to a proximal end part of the second operation member so as to move the second operation member to the insertion part.
